(19) United States
(12) Patent Application Publication
(10) Pub. No.: US 2024/0016548 A1
(43) Pub. Date: Jan. 18, 2024

(54) METHOD AND SYSTEM FOR MONITORING AN ORIENTATION OF A MEDICAL OBJECT

(71) Applicant: Siemens Healthcare GmbH, Erlangen (DE)

(72) Inventors: Marcus Pfister, Bubenreuth (DE); Markus Kowarschik, Nürnberg (DE)

(21) Appl. No.: 18/221,749

(22) Filed: Jul. 13, 2023

(30) Foreign Application Priority Data
Jul. 13, 2022 (DE) 10 2022 207 155.7

(51) Int. Cl. A61B 34/10; A61B 34/20; A61B 90/00; A61B 34/00

(52) U.S. Cl. CPC A61B 34/10; A61B 34/20; A61B 90/36; A61B 34/25; A61B 2034/107; A61B 2034/2055; A61B 2090/366; A61B 2090/376

(57) ABSTRACT

A method for monitoring an orientation of a medical object, includes identifying planning information having a planning orientation for the medical object with respect to a reference point on an anatomical object that is arranged within an examination object. The orientation of the medical object with respect to the reference point is detected using an acquisition unit. The acquisition unit includes a medical imaging device and/or an acoustic and/or optical and/or electromagnetic sensor. A deviation between the planning orientation and the orientation of the medical object with respect to the reference point is identified, and a signal is provided depending on the identified deviation.

This application claims the benefit of German Patent Application No. DE 10 2022 207 155.7, filed on Jul. 13, 2022, which is hereby incorporated by reference in its entirety.

### BACKGROUND

The present embodiments relate to a method and a system for monitoring an orientation of a medical object and a computer program product.

In minimally invasive interventions, therapies (e.g., placements of stents) or diagnoses (e.g., detection of stenoses) may be performed using medical objects inserted into the body. These medical objects may be advanced to their site of use through an access in the groin (e.g., the femoral artery) or the left axilla (e.g., radial access via the subclavian artery) using guide wires and catheters. Navigation into the individual vascular outlets may be accomplished by rotating and advancing the guide wire or catheter at the point of entry.

A first step is often the puncture as access for the medical objects. In the groin, this may be the common femoral artery, which is located about 2-5 cm under a skin of the examination object as the closest point to the surface about 1 cm to 2 cm distal to a groin ligament and next to a femoral head, the anterior tip of the hip bone. Although this artery is often easy to palpate, an incorrect puncture (e.g., one that is too steep) may adversely lead to complications (e.g., vascular perforation). These should be avoided at all costs, as bleeding may be potentially dangerous and cause prolonged recumbency of the examination object.

For example, in remote-controlled procedures (e.g., robotic remote procedures), the vascular punctures (e.g., the insertion of the medical object) are often performed on site. It is a disadvantage that a medical operator who remotely controls the robotic remote procedure may only monitor this procedure to a limited extent, which provides that in certain circumstances impending complications m be detected too late.

### SUMMARY AND DESCRIPTION

The scope of the present invention is defined solely by the appended claims and is not affected to any degree by the statements within this summary.

The present embodiments may obviate one or more of the drawbacks or limitations in the related art. For example, reliable monitoring of a puncture of an anatomical object by a medical object is provided.

The present embodiments relate, in a first aspect, to a method for monitoring an orientation of a medical object. In this case, planning information that has a planning orientation for the medical object with respect to a reference point on an anatomical object is identified. In this case, the anatomical object is arranged within an examination object. Further, the orientation of the medical object with respect to the reference point is detected by an acquisition unit. In this case, the acquisition unit includes a medical imaging device and/or an acoustic and/or optical and/or electromagnetic sensor. Further, a deviation between the planning orientation and the orientation of the medical object with respect to the reference point is identified. Further, a signal is provided in dependence upon the identified deviation.

The examination object may include, for example, a human and/or animal patient and/or an examination phantom (e.g., a vascular phantom). The anatomical object may include a hollow organ (e.g., a vascular portion, such as an artery and/or vein) and/or a lung and/or a heart and/or an organ (e.g., a liver) and/or a tissue (e.g., a tumor tissue) of the examination object. In this case, the anatomical object may be arranged at least in part (e.g., completely) within the examination object (e.g., below a skin surface of the examination object).

The medical object may, for example, be configured as a surgical and/or diagnostic instrument (e.g., an elongated instrument). For example, the medical object may be configured to be flexible and/or rigid at least in sections. The medical object may be configured, for example, as a needle (e.g., a puncture needle) and/or catheter and/or endoscope and/or guide wire.

Identifying the planning information may include receiving and/or determining the planning information (e.g., detecting the planning orientation with respect to the reference point). Receiving the planning information may include acquiring and/or reading a computer-readable data storage device and/or receiving from a data storage unit (e.g., a database). Further, the planning information may be provided by a provisioning unit of a medical device (e.g., the medical imaging device and/or a further medical imaging device). Alternatively or additionally, the planning information may be acquired based on user input from a medical operator. Alternatively or additionally, the planning information (e.g., the planning orientation with respect to the reference point) may be determined, for example, based on a user input and/or by applying a function (e.g., a trained function) to a planning map of the examination object with the anatomical object arranged therein.

The planning information may have the planning orientation for the medical object (e.g., at least one distal portion of the medical object) with respect to the reference point on the anatomical object. The distal portion of the medical object may include, for example, an end portion that is facing the examination object, and/or a tip of the medical object. In this case, the planning information may have a specification regarding the positioning (e.g., a spatial position) of the reference point on the anatomical object (e.g., with respect to the anatomical object). For example, the reference point may be specified (e.g., identified) with respect to a planning map of the examination object with the anatomical object arranged therein. In this case, the planning map may have a map (e.g., image data) and/or a representation (e.g., a model) of the anatomical object. In one embodiment, the reference point may be specified on a surface of the anatomical object (e.g., a vascular wall and/or a tissue boundary).

The planning orientation may have a specification regarding a planned orientation (e.g., a spatial positional relationship and/or pose and/or orientation) of the medical object with respect to the reference point (e.g., in the reference point), on the anatomical object. For example, the planning orientation may specify the planned orientation with respect to a predetermined plane (e.g., a tangential plane and/or a normal plane), through the reference point and with respect to the surface of the anatomical object. In this case, the planning information may have, in addition, information regarding the predetermined plane. For example, the planning information may specify the planning orientation for the medical object with respect to the reference point on the anatomical object in a coordinate system of the examination object. Further, the planning orientation may have a planning angle of a planned arrangement of the medical object in the reference point with respect to the predetermined plane.

In one embodiment, it is possible, using the acquisition unit, to detect the orientation (e.g., instantaneous and/or actual orientation; spatial positional relationship and/or pose and/or orientation) of the medical object with respect to the reference point. For this purpose, the acquisition unit may identify the reference point on the anatomical object (e.g., based on the planning information). In one embodiment, the acquisition unit may detect a relative positioning (e.g., instantaneous relative positioning) between the medical object and the reference point on the anatomical object. Further, the detected orientation may have information regarding a detected angle of the arrangement (e.g., instantaneous arrangement) of the medical object in the reference point with respect to the predetermined plane.

In one embodiment, the acquisition unit may include a medical imaging device. The medical imaging device may include, for example, a medical X-ray device (e.g., a medical C-arm X-ray device) and/or a computed tomography facility (CT facility) and/or a magnetic resonance tomography facility (MRT facility) and/or a positron emission tomography facility (PET facility) and/or an ultrasound device. Alternatively or additionally, the acquisition unit may include an acoustic (e.g., ultrasound-based) sensor. The acoustic sensor may be configured so as to detect the medical object (e.g., the orientation of the medical object with respect to the reference point) using acoustic localization. Alternatively or additionally, the acquisition unit may include an optical sensor (e.g., a camera, such as a mono camera and/or a stereo camera and/or depth camera). The optical sensor may be configured so as to optically detect the orientation of the medical object with respect to the reference point (e.g., based on image data). Alternatively or additionally, the acquisition unit may include an electromagnetic sensor (e.g., an electromagnetic localization system). The electromagnetic sensor may be configured so as to detect the orientation of the medical object with respect to the reference point using electromagnetic localization.

Identifying the deviation between the planning orientation and the orientation of the medical object with respect to the reference point may include registering the planning orientation with the detected orientation of the medical object (e.g., based on the reference point). The deviation may be identified as a distance between the distal portion (e.g., an end portion and/or a tip) of the medical object and the reference point, and/or as an angular difference between the planning angle specified by the planning orientation and the detected angle of the medical object with respect to the reference point.

In one embodiment, the signal may be provided in dependence upon the identified deviation (e.g., in dependence upon a presence of the deviation and/or a quantity of the deviation). In this case, the signal may be provided having information (e.g., qualitative and/or quantitative information) regarding the identified deviation.

The provision of the signal may include, for example, storage on a computer-readable storage medium and/or an output (e.g., visual and/or acoustic and/or haptic output) of the signal and/or a transmission to a provisioning unit.

The embodiment may enable reliable monitoring of the orientation of the anatomical object with respect to the reference point on the anatomical object (e.g., in the context of a puncture). For example, a medical operator may be assisted by the signal during orientating the medical object (e.g., in maintaining the planning orientation) with respect to the reference point. For example, the signal may be provided to an operator who is remotely controlling the medical object (e.g., a robot for guiding the medical object, such as a catheter robot).

In a further embodiment of the method, the planning information may have a planning map of the examination object with the anatomical object arranged therein. In this case, a planned entry point of the medical object into the anatomical object may be determined as the reference point. Further, the planning orientation may be determined in dependence upon a progression and/or an arrangement of the anatomical object, which is mapped in the planning map.

The planning map may have a two dimensional (2D) and/or three dimensional (3D) spatially resolved map (e.g., medical image data) of the examination object with the anatomical object arranged therein. Alternatively or additionally, the planning map may have a 2D and/or 3D spatially resolved representation (e.g., a model, such as a volume mesh model) of the examination object with the anatomical object arranged therein. In addition, the planning map may be time-resolved. In this case, the planning map may map the progression (e.g., a spatial position and/or orientation and/or pose of the anatomical object and/or a cavity of the anatomical object). Alternatively or additionally, the planning map may map the arrangement (e.g., spatial arrangement) of the anatomical object (e.g., a relative positioning of the anatomical object with respect to adjacent anatomical objects and/or a position of the reference point on the anatomical object).

The planned entry point (e.g., a penetration site and/or a puncture site) may be identified manually, semi-automatically, or fully automatically based on the planning map. For example, it is possible, using an input unit, to acquire a further user input that specifies the planned entry point with respect to the planning map. Alternatively or additionally, the planned entry point may be determined semi-automatically (e.g., within a predetermined spatial area) and/or fully automatically (e.g., based on geometric and/or anatomical features of the anatomical object that are mapped in the planning map). The semi-automatic and/or fully automatic determination of the entry point may be based, for example, on machine learning and/or artificial intelligence.

In one embodiment, the planned entry point may be determined as the reference point on the anatomical object. Further, the planning orientation may be determined in dependence upon the progression (e.g., a spatial position and/or orientation and/or pose) of the anatomical object and/or a cavity of the anatomical object. For example, the planning orientation may be determined in a tangential manner with respect to the progression of the anatomical object (e.g., a vascular portion of the anatomical object) in the reference point. Alternatively or additionally, the planning orientation may be determined in dependence upon an arrangement (e.g., spatial arrangement) of the anatomical object (e.g., a relative positioning of the anatomical object with respect to an anatomical object that is adjacent thereto and/or a position of the reference point on the anatomical object).

By taking into consideration the progression and/or the arrangement of the anatomical object during the determination of the planning orientation, it is possible to minimize a risk of injury for the examination object.

In a further embodiment of the method, a map of at least one anatomical landmark and/or at least one marker structure may be identified in the planning map. In this case, the reference point and/or the planning orientation may be determined in dependence upon an arrangement of the at least one anatomical landmark and/or at least one marker structure.

In one embodiment, it is possible to identify in the planning map the map of the at least one anatomical landmark (e.g., a plurality of anatomical landmarks) and/or at least one marker structure (e.g., a plurality of marker structures). The identification of the map of the at least one anatomical landmark and/or the at least one marker structure may be performed manually or automatically. For example, the map of the at least one anatomical landmark and/or the at least one marker structure may be identified (e.g., annotated) based on a user input from a medical operator. Alternatively, the map of the at least one anatomical landmark and/or the at least one marker structure may be identified automatically (e.g., by applying an algorithm for pattern recognition and/or a segmentation of the planning map, such as a threshold-based and/or contour-based segmentation). The at least one anatomical landmark may include, for example, a femoral head and/or a pelvis edge. The marker structure may include, for example, a contrast agent and/or a marker object (e.g., a representational marker object) that is visible on imaging, and/or a spatial (e.g., defined) arrangement of a plurality of marker objects. Identifying the map of the at least one anatomical landmark and/or the at least one marker structure in the planning map may further include identifying an arrangement (e.g., a spatial position and/or orientation and/or pose) of the at least one anatomical landmark and/or the at least one marker structure (e.g., in the coordinate system of the examination object).

In one embodiment, the reference point and/or the planning orientation may be determined in dependence upon (e.g., based on) the identified arrangement of the at least one anatomical landmark and/or the at least one marker structure. The determination of the reference point and/or the planning orientation may be performed manually (e.g., based on a user input) and/or automatically. The automatic determination of the reference point and/or the planning orientation may be based, for example, on a predefined positional relationship (e.g., a distance, such as a minimum distance) between the reference point and the at least one anatomical landmark and/or the marker structure and/or an arrangement of the planning orientation in a predetermined angle and/or angular range with respect to the at least one landmark and/or marker structure, and/or a risk assessment (e.g., with regard to a risk of perforation and/or bleeding). The at least one anatomical landmark (e.g., a plurality of anatomical landmarks) and/or the marker structure may define a geometric object (e.g., a plane and/or a polygon and/or a distance and/or a straight line). In this case, the reference point and/or the planning orientation may be determined in a defined positional relationship (e.g., in a predetermined distance and/or in a defined arrangement and/or a predetermined angle and/or tangential and/or parallel) with respect to the geometric object.

The embodiment may render possible an improved determination of the reference point and/or the planning orientation.

In a further embodiment of the method, intra-operative image data may be recorded by the medical imaging device. In this case, the intra-operative image data may have a map of a distal portion of the medical object that is arranged intra-operatively in the examination object. In this case, the orientation of the medical object with respect to the reference point may be detected based on the map of the distal portion of the medical object.

In one embodiment, the intra-operative image data may be recorded by the medical imaging device intra-operatively (e.g., while the distal portion of the medical object is arranged within the examination object). The intra-operative image data may have a 2D and/or 3D spatially resolved map of the distal portion of the medical object. In addition, the intra-operative image data may be time-resolved. The distal portion of the medical object may include, for example, an end portion that is facing the examination object, and/or a tip of the medical object. In one embodiment, the distal portion of the medical object may be arranged intra-operatively at the reference point. In one embodiment, the intra-operative image data may have, in addition, a map of the anatomical object (e.g., the reference point). In addition, the planning information may be registered with the intra-operative image data (e.g., in the coordinate system of the examination object and/or in a coordinate system of the medical imaging device).

Detecting the orientation of the medical object with respect to the reference point may include identifying the map of the distal portion of the medical object in the intra-operative image data. In this case, it is possible to identify image points (e.g., pixels and/or voxels) of the intra-operative image data. The image points map the distal portion of the medical object (e.g., using a pattern and/or object recognition and/or a segmentation, such as a threshold-based segmentation). In one embodiment, it is possible, in addition, to receive object information that has geometric features (e.g., a shape and/or curvature) and/or an operating parameter regarding the medical object. For example, the planning information may have the object information. In one embodiment, it is possible to detect the orientation of the medical object (e.g., a predetermined axis, such as a longitudinal axis) of the medical object, based on the map of the distal portion of the medical object and the object information. In this case, the object information may have, for example, information relating to a positional relationship of the predetermined axis with respect to the distal portion of the medical object.

In one embodiment, the intra-operative image data may be recorded repeatedly (e.g., until the occurrence of a termination condition) using the medical imaging device. In this case, the orientation of the medical object with respect to the reference point may be repeatedly detected based on the map (e.g., last recorded map) of the distal portion of the medical object. Further, the deviation between the planning orientation and the orientation of the medical object with respect to the reference point may be repeatedly identified, and the signal may be provided in dependence upon the identified deviation.

Based on the intra-operative image data, the embodiment may render possible a reliable monitoring of the orientation of the medical object with respect to the reference point.

In a further embodiment of the method, the medical imaging device may include an X-ray source and a detector that are arranged in a defined arrangement with respect to one another. In this case, the medical imaging device may also have a light-guiding facility that is arranged on the X-ray source. Further, it is possible, using the light-guiding facility, to project onto a surface of the detector a light pattern, having at least one straight line, so as to indicate a detector reference point. In this case, the defined arrangement of X-ray source and detector may be repositioned based on the planning information, such that the reference point of the anatomical object is arranged on a beam from the X-ray source to the detector reference point and a projection of the planning orientation onto the surface of the detector corresponds to the at least one projected straight line.

The X-ray source may be configured so as to emit X-ray beams (e.g., to emit a bundle of X-rays). Further, the detector (e.g., an X-ray detector) may be configured to receive the X-ray beams (e.g., the bundle of X-rays) after an interaction with the examination object. In one embodiment, the X-ray source and the detector may be arranged in a defined arrangement with respect to one another (e.g., on a C-arm and/or at least a robotic arm and/or a stand). The X-ray source may illuminate the examination object using X-ray beams that are received by the detector after the interaction with the examination object. Based on the received X-ray beams, it is possible to provide, for example, the intra-operative image data.

The medical imaging device may also have the light-guiding facility that is arranged on the X-ray source. For example, the light-guiding facility may be arranged on the X-ray source in a defined arrangement (e.g., fixedly and/or in a defined movable manner, such as attached) and/or at least in part integrated in the X-ray source. The light-guiding facility may include a light source (e.g., a laser light source) that projects the light pattern (e.g., predetermined light pattern) onto the surface of the detector. For this purpose, the light-guiding facility (e.g., a light source) may emit a predetermined light distribution (e.g., a predetermined distribution of laser light). In this case, the predetermined light distribution may project the light pattern, having the at least one straight line, onto the detector.

In one embodiment, the detector reference point may be arranged on a surface of the detector. The surface may be illuminated by the X-ray beams. In this case, the detector reference point may have a defined positioning with respect to the surface of the detector (e.g., with respect to a boundary region of the surface of the detector). For example, the detector reference point may mark a geometric middle point of the surface (e.g., X-ray beam sensitive surface) of the detector. In one embodiment, the light pattern may display the detector reference point on the surface of the detector. For example, an end point of the at least one straight line may mark (e.g., indicate) the detector reference point.

In one embodiment, the defined arrangement of X-ray source and detector may be repositioned (e.g., moved) in a translational and/or rotational manner, based on the planning information such that the reference point of the anatomical object is arranged on the beam from the X-ray source to the detector reference point. This renders it possible to provide that the reference point on the anatomical object is mapped in the intra-operative image data, which may be recorded by the medical imaging device (e.g., the defined arrangement of X-ray source and detector).

In addition, the defined arrangement of X-ray source and detector may be repositioned (e.g., moved) in a translational and/or rotational manner, based on the planning information, such that the at least one straight line that is projected by the light-guiding facility (e.g., one straight line of a plurality of projected straight lines) corresponds to (e.g., coincides with) the projection (e.g., virtual projection) of the planning orientation onto the surface of the detector. In one embodiment, the planning orientation may be projected along a projection direction of the light distribution virtually onto the surface of the detector (e.g., as shadowing).

By virtue of the fact that the reference point of the anatomical object is arranged on the beam from the X-ray source to the detector reference point and the projection of the planning orientation onto the surface of the detector corresponds to the at least one projected line, both the entry point and also the planning orientation may be displayed for the medical object using the light pattern.

The embodiment may assist (e.g., visually guide) a medical operator during the arrangement of the medical object on the reference point and along the planning orientation. In addition, by arranging the reference point on the beam from the X-ray source to the detector reference point, it is possible to provide the imaging-based monitoring of the orientation of the medical object.

In a further embodiment of the method, the light pattern may have a further geometric object that is arranged on the at least one straight line. In this case, a point of intersection of the at least one straight line with the further geometric object may indicate the detector reference point.

The further geometric object may include, for example, a point and/or a pattern and/or a cross and/or an arrow and/or a further line (e.g., straight line). In one embodiment, a combination of the at least one straight line and further geometric object may be projected as the light pattern onto the surface of the detector.

In one embodiment, the further geometric object may be arranged on the at least one straight line (e.g., intersecting the at least one straight line at the point of intersection). In this case, the projected point of intersection of the at least one straight line and the further object may indicate the detector reference point (e.g., the beam from the X-ray source to the detector reference point).

As a consequence, it is possible, using the point of intersection, to indicate the reference point of the anatomical object, which is arranged on a beam from the X-ray source to the detector reference point.

In a further embodiment of the method, it is possible, busing the acquisition unit, to detect a positioning of at least one proximal portion of the medical object, which is arranged intra-operatively outside the examination object. In this case, the orientation of the medical object with respect to the reference point may be detected based on the positioning of the proximal portion.

The proximal portion of the medical object may include a portion of the medical object that is remote from the examination object (e.g., is facing a medical operator). Further, the proximal portion may be arranged intra-operatively outside (e.g., extra corporeal) of the examination object. In one embodiment, the sensor may detect the positioning (e.g., the spatial position and/or orientation and/or pose) at least of the proximal portion (e.g., in addition to the distal portion and/or the entire medical object).

In one embodiment, the positioning of the proximal portion of the medical object may be detected by the sensor and/or or the medical imaging device. If the sensor is configured as an acoustic sensor (e.g., an ultrasound-based sensor), the orientation of the proximal portion of the medical object may be detected using acoustic localization. If the sensor is configured as an optical sensor, the positioning of the proximal portion of the medical object may be optically detected (e.g., based on image data). If the sensor is configured as an electromagnetic sensor, the orientation of the proximal portion of the medical object may be detected using electromagnetic localization.

Alternatively or additionally, the positioning of the proximal portion may be detected based on image data (e.g., the intra-operative image data) that is recorded intra-operatively by the medical imaging device.

In one embodiment, it is possible, in addition, to receive the object information that has geometric features (e.g., a shape and/or curvature) and/or an operating parameter regarding the medical object. For example, the planning information may have the object information. In one embodiment, the object information may describe a positional relationship between the proximal portion of the medical object and the distal portion of the medical object (e.g., a straight-line arrangement). In this case, the orientation of the medical object with respect to the reference point may be detected based on the detected positioning of the proximal portion and the object information (e.g., the positional relationship between the proximal and the distal portion of the medical object).

The embodiment may render possible an extra corporeal detection of the orientation (e.g., instantaneous orientation) of the medical object with respect to the reference point.

In a further embodiment of the method, it is possible to detect a positioning of the examination object and/or the anatomical object using the acquisition unit. In this case, the planning information may be registered with the detected positioning of the examination object and/or the anatomical object.

The positioning (e.g., instantaneous positioning; a spatial position and/or orientation and/or pose) of the anatomical object may be detected using the acquisition unit. Alternatively or additionally, the positioning of the examination object may be detected using the acquisition unit. In this case, the positioning of the anatomical object may be determined based on the detected positioning of the examination object and based on information regarding a relative positioning of the anatomical object with respect to the examination object. The relative positioning may be predetermined, for example, based on pre-procedural data (e.g., image data and/or a model) of the examination object and/or a patient model (e.g., a generic patient model).

Detecting the positioning of the examination object and/or the anatomical object may be performed by the medical imaging device and/or the sensor for detecting the orientation of the medical object and/or a further sensor (e.g., acoustic and/or optical and/or electromagnetic and/or mechanical sensor).

In one embodiment, the planning information (e.g., the planning orientation) and/or the reference point may be registered with the detected positioning of the examination object and/or the anatomical object. Registering the planning information with the detected positioning of the examination object and/or anatomical object may be based, for example, on geometric and/or anatomical features, the positioning of which is both identified in the planning information and determined based on the detected positioning. The geometric features may include, for example, a contour and/or an edge and/or surface and/or shape of the examination object and/or the anatomical object and/or a marker structure. Further, the anatomical features may include, for example, an anatomical landmark and/or a tissue boundary.

In addition, it is possible, based on the detected positioning of the examination object and/or the anatomical object, to detect a positioning (e.g., an instantaneous positioning) of the reference point on the anatomical object. In one embodiment, it is possible to provide the registered planning information (e.g., the registered planning orientation). For example, it is possible to provide the registered planning orientation with respect to the detected positioning (e.g., instantaneous positioning) of the reference point.

In a further embodiment of the method, providing the signal may include outputting a visual and/or acoustic and/or haptic warning signal.

In one embodiment, it is possible in dependence upon the identified deviation (e.g., upon a predetermined threshold value with respect to the deviation being reached or exceeded) to output the visual and/or acoustic and/or haptic warning signal (e.g., using an output unit). The visual warning signal may include, for example, a light signal and/or a graphic representation. Further, the acoustic warning signal may include a sound (e.g., a voice output) and/or an output of a sound sequence. In addition, the haptic warning signal may include a vibration. In one embodiment, it is possible, using the visual and/or acoustic and/or haptic warning signal, to indicate the deviation (e.g., upon the predetermined threshold value with respect to the deviation being reached or exceeded). In this case, the warning signal may indicate the deviation in a qualitative and/or quantitative manner. For example, the warning signal may be adapted (e.g., modulated) in dependence upon the deviation.

It is possible by providing the warning signal to assist the medical operator during the arrangement of the medical object along the planning orientation with respect to the reference point (e.g., issue a warning in the case of an identification of a deviation). For example, the warning signal may be provided to an operator who is remotely controlling the medical object (e.g., a robot for guiding the medical object, such as a catheter robot).

In a further embodiment of the method, providing the signal may include outputting a workflow instruction so as to minimize the deviation.

In one embodiment, providing the signal may include outputting a workflow instruction (e.g., an, visual and/or acoustic and/or haptic). The visual workflow instruction may include, for example, a light signal and/or a graphic representation. Further, the acoustic workflow instruction may include a sound output (e.g., a voice output) and/or an output of a sound sequence. In addition, the haptic workflow instruction may include a vibration. In this case, the workflow instruction may include a, for example, quantitative and/or qualitative specification so as to minimize the deviation. For example, the specification may indicate a direction from an instantaneous orientation of the medical object to the planning orientation and/or a distance between the instantaneous orientation of the medical object and the planning orientation. In addition, the workflow instruction may be adapted in dependence upon the deviation.

Using the output workflow instruction, the embodiment may assist the medical operator during the correction of the identified deviation with respect to the planning orientation.

In a further embodiment of the invention, the identified deviation may be compared with a predetermined threshold value. In this case, the signal may be provided upon the threshold value being reached and/or exceeded.

In one embodiment, the planning information may include the predetermined threshold value. Alternatively or additionally, the threshold value may be acquired based on user input from a medical operator (e.g., using an input unit). In one embodiment, the identified deviation between planning orientation and the orientation of the medical object with respect to the reference point may be compared with the predetermined threshold value. Comparing the identified deviation with the predetermined threshold value may include determining a difference and/or a quotient. In this case, the signal may be provided upon (e.g., only upon) the threshold value being reached and/or exceeded by the deviation.

The present embodiments relate, in a second aspect, to a system for monitoring an orientation of a medical object. In this case, the system includes an acquisition unit and a provisioning unit. The provisioning unit is configured so as to identify planning information, having a planning orientation for the medical object with respect to a reference point on an anatomical object that is arranged within an examination object. The acquisition unit is configured so as to detect the orientation of the medical object with respect to the reference point. The acquisition unit includes a medical imaging device and/or an acoustic and/or optical and/or electromagnetic sensor. The provisioning unit is configured so as to identify a deviation between the planning orientation and the orientation of the medical object with respect to the reference point. Further, the provisioning unit is configured so as to provide a signal in dependence upon the identified deviation.

The advantages of the system of the present embodiments correspond essentially to the advantages of the method of the present embodiments for monitoring an orientation of a medical object. In so doing, mentioned features, advantages, or alternative embodiments may likewise also be transferred to other subject matters and conversely.

In a further embodiment of the system, the medical imaging device may be configured so as to record intra-operative image data. In this case, the intra-operative image data may have a map of a distal portion of the medical object that is arranged in an operational state of the system in the examination object. Further, the provisioning unit may be configured so as to detect the orientation of the medical object with respect to the reference point based on the map of the distal portion of the medical object.

In a further embodiment of the system, the medical imaging device may include an X-ray source and a detector that are arranged in a defined arrangement with respect to one another. In this case, the medical imaging device may also have a light-guiding facility that is arranged on the X-ray source and configured so as to project onto a surface of the detector a light pattern, having at least one straight line, so as to indicate a detector reference point. In addition, the defined arrangement of X-ray source and detector may be repositioned in the operational state based on the planning information such that the reference point of the anatomical object is arranged on a beam from the X-ray source to the detector reference point and a projection of the planning orientation onto the surface of the detector corresponds to the at least one projected straight line.

In a further embodiment of the system, the system may also include an apparatus for the robotic remote manipulation of the medical object (e.g., a catheter robot). In this case, the provisioning unit may be configured so as to provide the signal to the apparatus.

In one embodiment, the apparatus is arranged in the operational state outside the examination object. Further, the apparatus may have an attachment element (e.g., movable and/or traversable attachment element). In addition, the apparatus may have a cassette element that is configured so as to record the proximal portion of the medical object. Further, the apparatus may have a movement element that is attached to the attachment element (e.g., a stand and/or robotic arm). In addition, the attachment element may be configured so as to attach the movement element to a patient positioning apparatus. Further, the movement element may have at least one actuator element (e.g., an electric motor that may be controlled by the provisioning unit). In one embodiment, the cassette element may be coupled (e.g., in a mechanical and/or electromagnetic and/or pneumatic manner) to the movement element (e.g., the at least one actuator element). In this case, the cassette element may also have at least one transmission element that may be moved by the coupling between the cassette element and the movement element (e.g., the at least one actuator element). For example, the at least one transmission element may be movement-coupled to the at least one actuator element. In one embodiment, the transmission element may be configured so as to transmit a movement of the actuator element to the medical object such that the medical object is moved along a longitudinal direction of extent of the medical object and/or that the medical object is rotated about its longitudinal direction of extent. The at least one transmission element may have, for example, a roller and/or a drum and/or shutter or shear plate. Further, the transmission element may be configured so as to hold the medical object (e.g., in a stable manner) by transmitting a force. Holding the medical object may include, for example, fixedly positioning at least the medical object with respect to the apparatus. In one embodiment, the movement element may have a plurality of actuator elements (e.g., a plurality of independently controllable actuator elements). Further, the cassette element may have a plurality of transmission elements (e.g., for each of the actuator elements) at least one movement-coupled transmission element. As a consequence, it may render possible a movement (e.g., independent and/or simultaneous movement) of the medical object along different degrees of freedom of movement.

The apparatus may also include the acoustic and/or optical and/or electromagnetic sensor that is configured so as to detect the orientation of the medical object (e.g., a spatial positioning of the proximal portion of the medical object). In one embodiment, the system (e.g., the apparatus) may also include an input unit that is configured so as to acquire a user input from an operator who is remotely controlling the apparatus. In this case, the apparatus may be configured so as to adapt the positioning and/or movement of the medical object in dependence upon the user input. In addition, the system (e.g., the apparatus) may include the output unit that may be configured to output the warning signal to the operator.

The embodiment may render possible a reliable monitoring of the orientation of the medical object even when the procedure is being remotely controlled by the apparatus.

The present embodiments relate, in a third aspect, to a computer program product having a computer program that may be loaded directly into a storage device of a provisioning unit, having program steps in order to perform all the steps of a method for monitoring an orientation of a medical object if the program steps are performed by the provisioning unit.

Further, the present embodiments may relate to a computer-readable storage medium on which are stored program steps that may be read and performed by a provisioning unit in order to perform all the steps of the method for monitoring an orientation of a medical object if the program steps are performed by the processing unit.

A largely software-based realization has the advantage that provisioning units already in use may be easily retrofitted by a software update in order to operate in the manner according to the present embodiments. Such a computer program product may include, in addition to the computer program, where appropriate, additional components, such as, for example, documentation and/or additional components, as well as hardware components, such as, for example, hardware keys (e.g., dongles, etc.) for using the software.

### BRIEF DESCRIPTION OF THE DRAWINGS

Exemplary embodiments of the invention are illustrated in the drawings and are further described below. Same reference characters are used for same features in the different figures. In the drawings:

FIGS. 1 and 2 show schematic representations of different embodiments of a method for monitoring an orientation of a medical object.

FIGS. 3 to 5 show schematic representations in each case of a planning map.

FIGS. 6 to 8 show schematic representations of further embodiments of a method for monitoring an orientation of a medical object.

FIGS. 9 to 11 show schematic representations of different embodiments of a system for monitoring an orientation of a medical object.

FIG. 12 shows a schematic representation of a projected light pattern in an operational state of the system.

### DETAILED DESCRIPTION

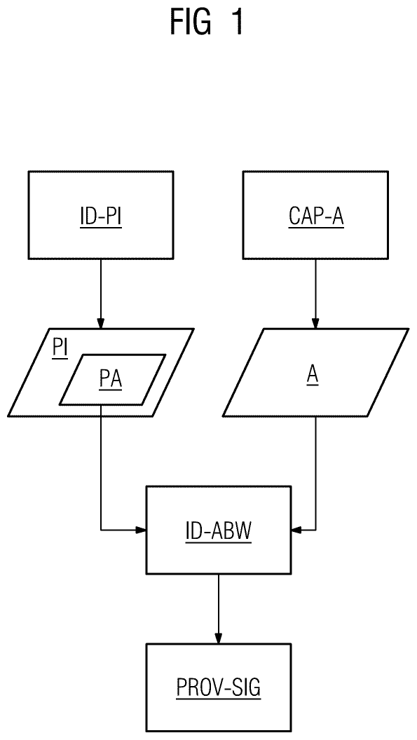

FIG. 1 illustrates schematically one embodiment of a method for monitoring an orientation A of a medical object. In this case, planning information PI having a planning orientation PA for the medical object may be identified ID-PI with respect to a reference point on an anatomical object that is arranged within an examination object. Further, the orientation A of the medical object with respect to the reference point may be detected CAP-A by an acquisition unit. In this case, the acquisition unit may include a medical imaging device and/or an acoustic and/or optical and/or electromagnetic sensor. In addition, it is possible to identify ID-ABW a deviation between the planning orientation PA and the orientation A of the medical object with respect to the reference point. In this case, the identified deviation may be compared with a predetermined threshold value. Accordingly, a signal may be provided PROV-SIG in dependence upon the identified deviation (e.g., upon the threshold value being reached and/or exceeded).

In this case, it is possible, using the acquisition unit, to detect at least one positioning of a proximal portion of the medical object that is arranged intra-operatively outside the examination object. Further, the orientation A of the medical object with respect to the reference point may be detected CAP-A based on the positioning of the proximal portion.

In one embodiment, providing the signal PROV-SIG may include outputting a visual and/or acoustic and/or haptic warning signal. Further, providing the signal PROV-SIG may include outputting a workflow instruction so as to minimize the deviation.

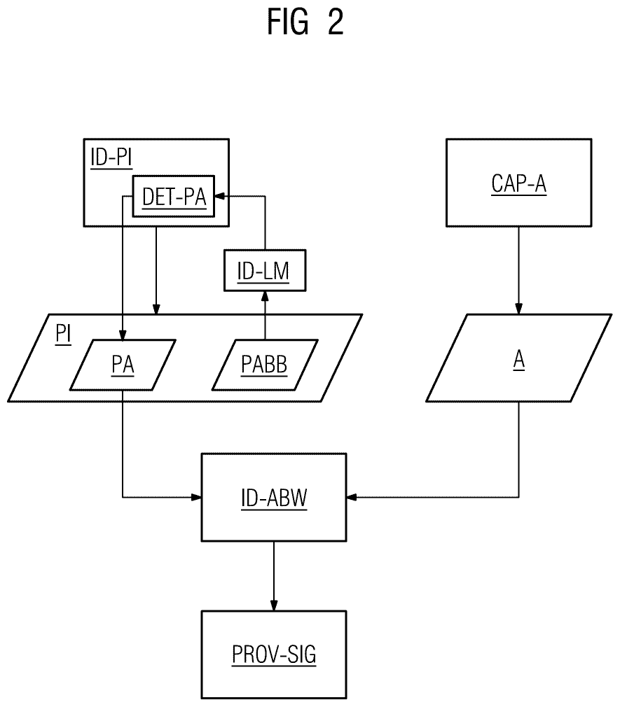

FIG. 2 shows a schematic representation of a further embodiment of the method for monitoring an orientation A of a medical object. In this case, the planning information PI may have a planning map PABB of the examination object with the anatomical object arranged therein. In one embodiment, a planned entry point of the medical object into the anatomical object may be determined as the reference point. In this case, the planning orientation PA may be determined DET-PA in dependence upon a progression and/or an arrangement of the anatomical object that is mapped in the planning map. Further, a map at least of one anatomical landmark and/or at least one marker structure may be identified ID-LM in the planning map PABB. In this case, the reference point and/or the planning orientation PA may be determined DET-PA in dependence upon an arrangement of the at least one anatomical landmark and/or the at least one marker structure.

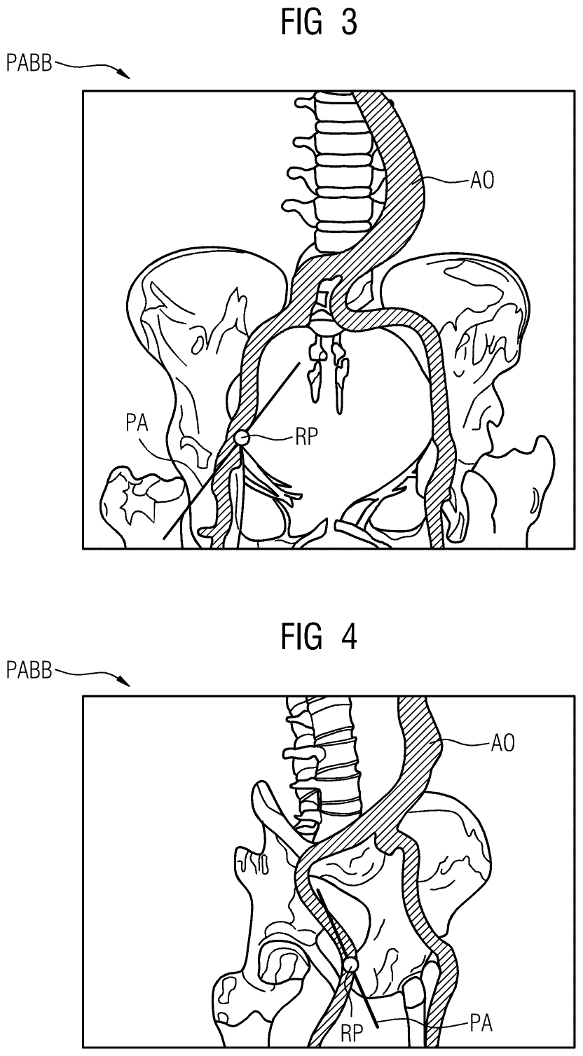

FIGS. 3 and 4 illustrate schematically a planning map PABB that has in each case a map of the anatomical object AO from different mapping directions. In this case, the anatomical object may include a vascular portion (e.g., an artery or vein). In one embodiment, the planned entry point of the medical object into the anatomical object AO may be determined as the reference point RP. In this case, the planning orientation PA may be determined DET-PA in dependence upon the progression and/or the arrangement of the anatomical object AO that is mapped in the planning map PABB.

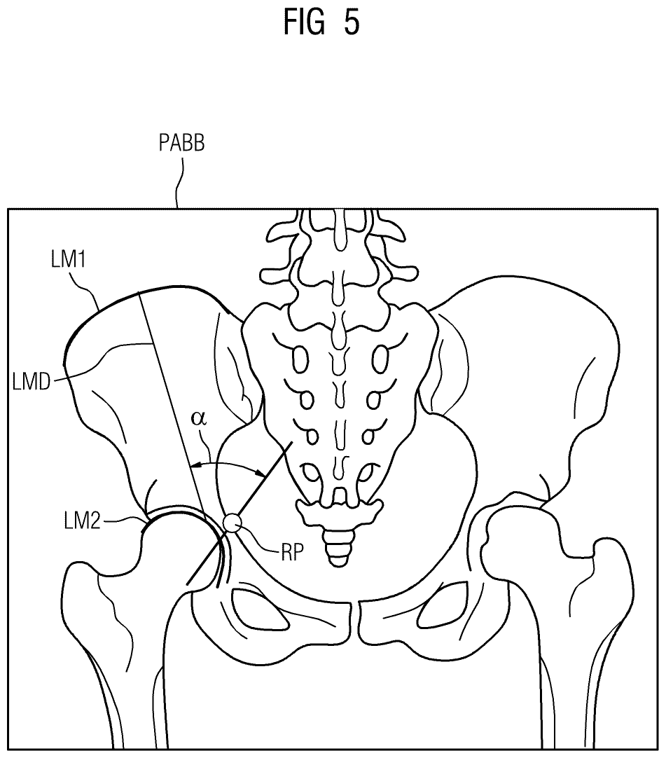

FIG. 5 shows a further schematic representation of a planning map PABB that has a map of two anatomical landmarks LM1 and LM2. In this case, the first landmark LM1 may include a pelvis edge, and the second landmark LM2 may include a femoral head of the examination object 31. In one embodiment, the reference point RP and the planning orientation PA may be determined DET-PA in dependence upon the arrangement (e.g., the relative positioning) of the two anatomical landmarks LM1 and LM2. The relative positioning of the two anatomical landmarks LM1 and LM2 may be characterized, for example, by a connecting section LMD. In this case, the planning orientation PA may be determined DET-PA in a predetermined angle α with respect to the connecting section LMD.

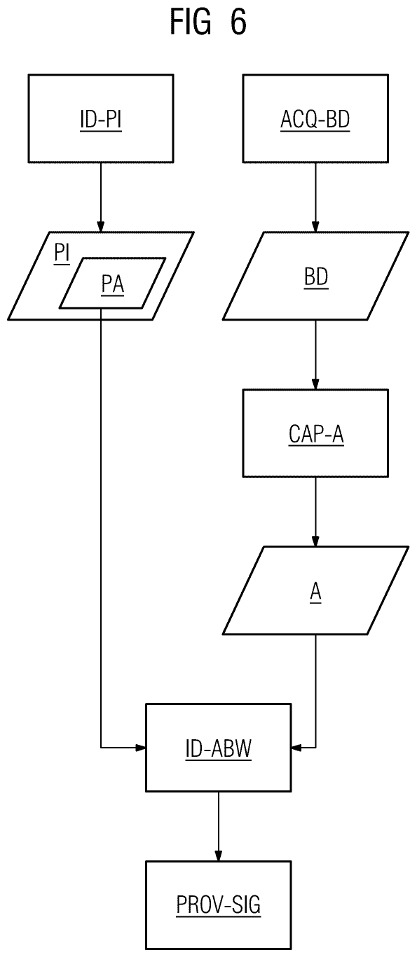

FIG. 6 illustrates schematically a further embodiment of the method for monitoring an orientation A of a medical object. In this case, intra-operative image data BD may be recorded by the medical imaging device ACQ-BD. The intra-operative image data BD may have a map of a distal portion of the medical object that is arranged intra-operatively in the examination object. In addition, the orientation A of the medical object with respect to the reference point may be detected CAP-A based on the map of the distal portion of the medical object.

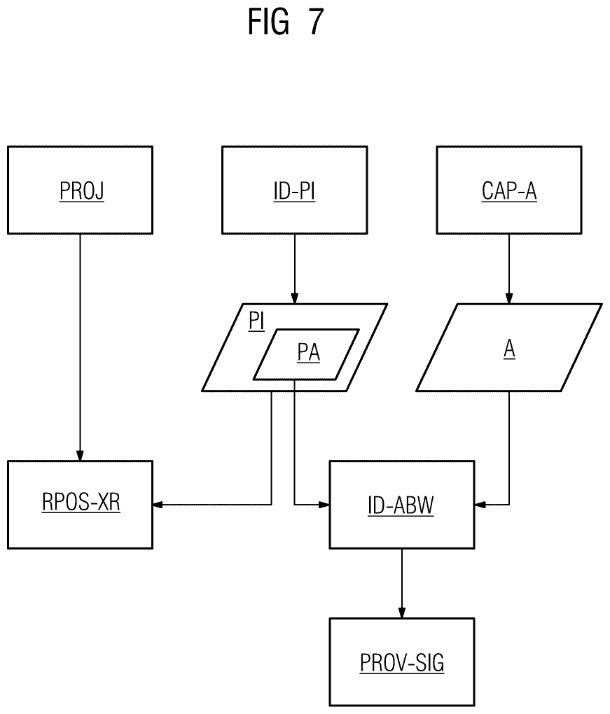

FIG. 7 shows a schematic representation of a further embodiment of a method for monitoring an orientation A of a medical object. In this case, the medical imaging device may include an X-ray source and a detector that are arranged in a defined arrangement with respect to one another. Further, the medical imaging device may have a light-guiding facility that is arranged on the X-ray source. In this case, it is possible, using the light-guiding facility, to project PROJ onto a surface of the detector a light pattern, having at least one straight line, so as to indicate a detector reference point. In one embodiment, the defined arrangement of X-ray source and detector may be repositioned based on the planning information PI such that the reference point of the anatomical object is arranged on a beam from the X-ray source to the detector reference point, and a projection of the planning orientation onto the surface of the detector corresponds to the at least one projected straight line. In one embodiment, the light pattern may have a further geometric object that is arranged on the at least one straight line. In this case, a point of intersection of the at least one straight line with the further geometric object may indicate the detector reference point.

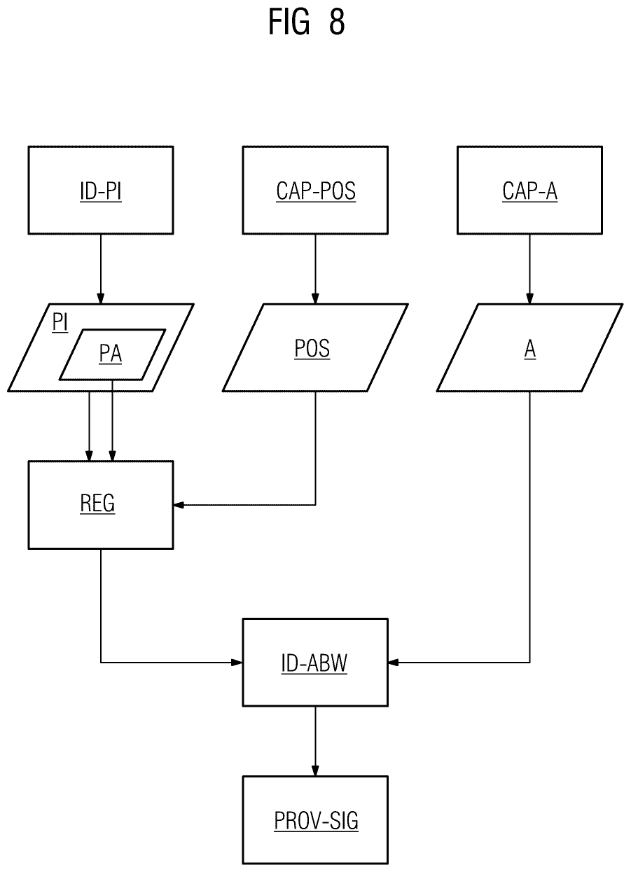

FIG. 8 shows a further embodiment of the method for monitoring an orientation A of a medical object. In one embodiment, a positioning POS of the examination object and/or the anatomical object may be detected CAP-POS using the acquisition unit. In this case, the planning information PI may be registered REG with the detected positioning POS of the examination object and/or the anatomical object.

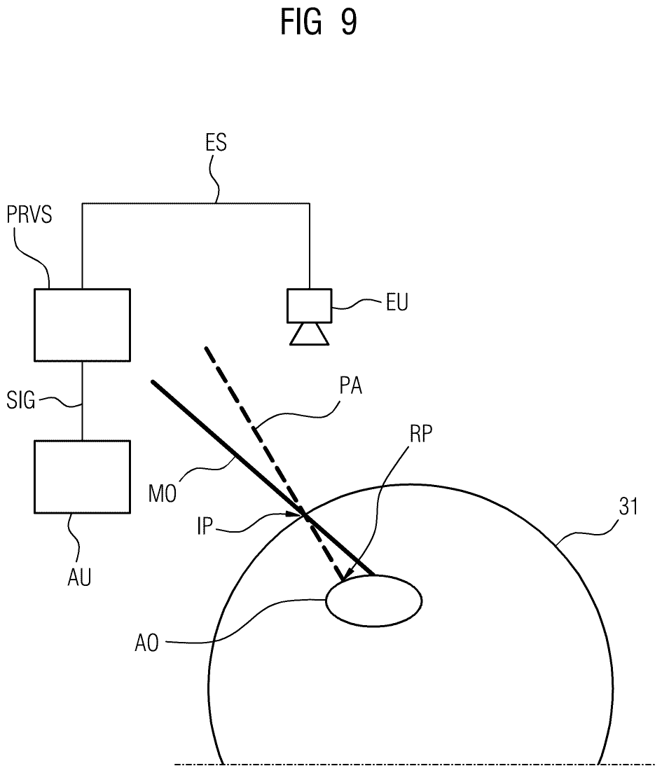

FIG. 9 shows a schematic representation of an embodiment of a system for monitoring an orientation A of a medical object. In this case, the system includes the acquisition unit EU and a provisioning unit PRVS. The provisioning unit PRVS may be configured so as to identify ID-PI the planning information PI, having the planning orientation PA for the medical object MO with respect to the reference point RP on the anatomical object AO that is arranged within an examination object 31 via an entry point IP. Further, the acquisition unit EU is configured so as to detect the orientation A of the medical object MO with respect to the reference point RP. The acquisition unit EU may include an acoustic and/or optical and/or electromagnetic sensor. In one embodiment, the provisioning unit PRVS may be configured so as to identify the deviation ID-ABW between the planning orientation PA and the orientation A of the medical object with respect to the reference point RP. In addition, the provisioning unit PRVS may be configured so as to provide PROV-SIG the signal SIG in dependence upon the identified deviation. For example, it is possible, using an output unit AU (e.g., a loudspeaker and/or a representation unit and/or a haptic signal transmitter), to output a visual and/or acoustic and/or haptic warning signal in dependence upon the signal SIG.

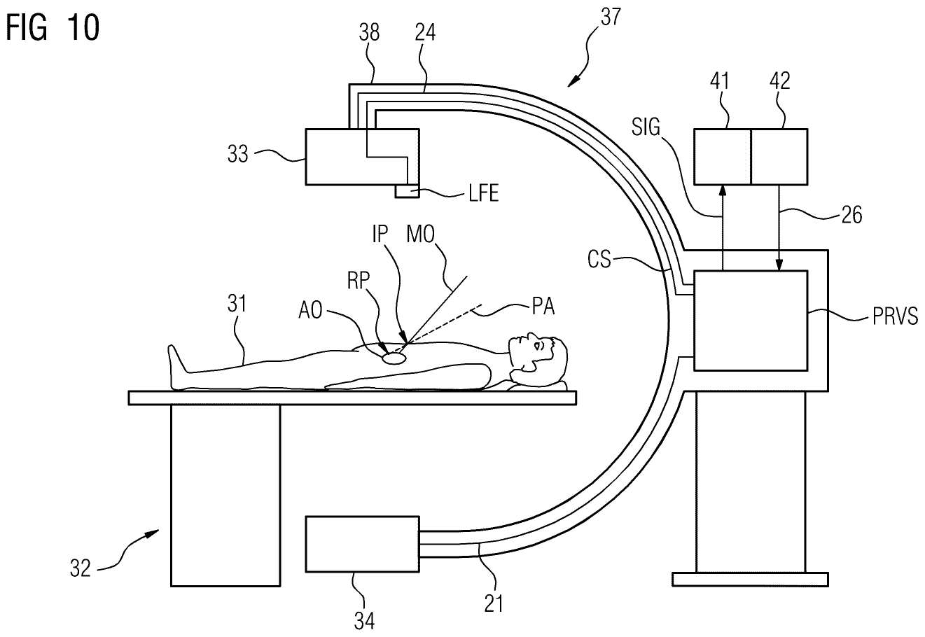

FIG. 10 illustrates schematically a further embodiment of the system for monitoring an orientation A of a medical object MO. In this case, the acquisition unit EU may include a medical imaging device (e.g., a medical C-arm X-ray device 37). The medical C-arm X-ray device 37 may have a detector 34 (e.g., an X-ray detector) and an X-ray source 33 that are arranged on the C-arm 38 in a defined arrangement with respect to one another. In order to record ACQ-BD intra-operative image data BD of the examination object 31, the provisioning unit PRVS may transmit a signal 24 to the X-ray source 33. Subsequently, the X-ray source 33 may emit a bundle of X-ray beams. When the bundle of X-ray beams impinges on the surface of the detector 34, after an interaction with the examination object 31, the detector 34 may transmit a signal 21 to the provisioning unit PRVS. The provisioning unit PRVS may receive the intra-operative image data BD based on the signal 21. The provisioning unit PRVS may be configured so as to detect CAP-A the orientation A of the medical object MO with respect to the reference point RP based on the map of the distal portion of the medical object MO in the intra-operative image data BD.

In one embodiment, the medical imaging device (e.g., the medical C-arm X-ray device 37) may also have a light-guiding facility LFE that is arranged on the X-ray source 33. In this case, the light-guiding facility LFE may be configured so as to project PROJ onto a surface of the detector a light pattern, having at least one straight line, 34 so as to indicate a detector reference point. In addition, the defined arrangement of X-ray source 33 and detector 34 may be repositioned RPOS-XR in the operational state of the system based on the planning information PI such that the reference point RP of the anatomical object AO is arranged on a beam from the X-ray source 33 to the detector reference point and the projection of the planning orientation PA onto the surface of the detector 34 corresponds to the at least one projected straight line. The provisioning unit PRVS may control the light-guiding facility LFE using a signal CS so as to project PROJ the light pattern.

Further, the system may have a representation unit 41 and an input unit 42. The representation unit 41 may have, for example, a monitor and/or a display and/or a projector. The input unit 42 may include, for example, a keyboard and/or a pointing device. The input unit 42 may, in one embodiment, be integrated into the display unit 41 (e.g., in the case of a capacitive and/or resistive input display). The input unit 42 may be configured so as to acquire a user input. Further, the input unit 42 may transmit a signal 26 to the provisioning unit PRVS. The provisioning unit PRVS may be configured so as to control the medical C-arm X-ray device 37 and/or the light-guiding facility LFE in dependence upon the user input (e.g., in dependence upon the signal 26). In addition, the provisioning unit PRVS may provide PROV-SIG the signal SIG to the representation unit 41. In this case, the representation unit 41 may be configured so as to display a graphic representation of the deviation and/or the warning signal (e.g., based on the signal SIG). Further, the provisioning unit may be configured so as to transmit a further signal 25 to the representation unit 41, where the representation unit 41 may be configured so as, based on the signal 25, to display a graphic representation of the planning information PI (e.g., the planning map PABB and/or the intra-operative image data BD).

Using the example of a puncture of a groin (e.g., via the femoral artery) as the anatomical object, it is possible to illustrate below an embodiment of the method (e.g., using a system of the present embodiments). The method may also assist a medical operator in a similar manner during puncturing radial accesses. Based on the planning map PABB (e.g., pre-operative 3D image data and/or intra-operative 2D image data) of the examination object 31 with the anatomical object AO, it is possible to plan the puncture of the groin (e.g., automatically), and to identify ID-PI the planning information PI having the planning orientation PA. In this case, it is possible to determine the reference point (e.g., the entry point IP, such as a puncture site and/or a penetration point) of the medical object MO into the anatomical object AO, either based on the pre-operative 3D image data and/or based on the intra-operative 2D image data (e.g., 2D fluoroscopic images). During planning based on the pre-operative 3D image data, it is initially possible to determine the penetration site as the reference point RP based on a progression of the anatomical object AO (e.g., a vascular structure), and subsequently to determine an optimum puncture direction (e.g., tangential to a local vascular structure) as the planning orientation PA. In order to improve the orientation, it is possible in this case to identify the planning orientation PA (e.g., a 3D puncture path) separated into two spatial angles parallel and perpendicular to a longitudinal axis of the patient positioning apparatus 32. In the case of an identification of the planning information PI based on intra-operative 2D image data, it is possible to determine the reference point (e.g., the penetration site) and the planning orientation PA (e.g., a penetration angle), for example, based on the at least one anatomical landmark and/or the at least one marker structure (e.g., the femoral head and/or the pelvis edge). In this case, for example, only a graphic representation of the optimum puncture angle parallel to a longitudinal axis of the patient positioning apparatus 32 may be displayed by the representation unit 41.

Further, it is possible to identify the deviation between the orientation A (e.g., actual orientation) of the medical object MO during puncturing and the planning orientation PA. During the puncture procedure, it is possible to identify a map of the medical object MO (e.g., a needle) in the intra-operative image data BD (e.g., a 2D fluoroscopic image), and compare the orientation A with the planning orientation PA. In this case, the planning orientation PA may either be a path that is calculated from the intra-operative 2D image data and/or a 3D path that is forwards projected from the pre-operative 3D image data, where, for this purpose, the pre-operative 3D image data is registered using a coordinate system of the medical C-arm X-ray device 37. In the case of an excessively large deviation between the detected orientation A of the medical object MO and the planning orientation PA (e.g., upon the predetermined threshold value being reached or exceeded), it is possible to warn the medical operator by providing a warning signal. In the case of a remote-controlled procedure, it is possible, in addition to warning the local operator, to also warn a remote-controlling operator (e.g., a “remote operator”). The operator may be notified of the deviation, for example, by outputting the acoustic and/or visual and/or haptic warning signal. Alternatively or additionally, it is possible to display to the operator, using the representation unit 31, a graphic representation of the identified deviation (e.g., a determined deviation value). In addition, providing the signal PROV-SIG may include outputting the workflow instruction (e.g., a graphic representation of the workflow instruction, such as a correction proposal), so as to minimize the deviation.

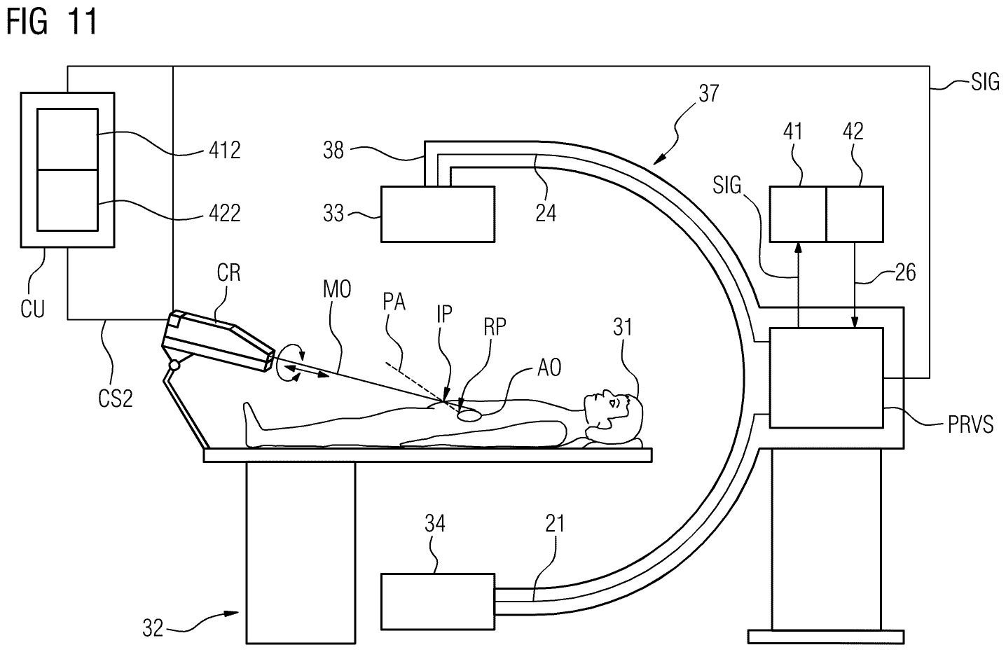

FIG. 11 illustrates schematically a further embodiment of the system. In this case, the system may further include an apparatus CR for the robotic remote manipulation of the medical object MO. The distal portion of the medical object MO may be arranged in the operational state of the system at least in part in the examination object 31. Further, the apparatus CR may be attached (e.g., in a movable manner) to the patient positioning apparatus 32 using an attachment element (e.g., a stand and/or robotic arm). As a consequence, it is possible to predetermine a spatial positioning of the proximal portion of the medical object MO, which is arranged at least in part in the apparatus CR, with respect to the examination object. In one embodiment, the apparatus CR may be configured so as to move the medical object MO, which is arranged in the operational state of the system at least in part in the apparatus CR, in a translational manner at least along a longitudinal direction of extent of the medical object MO. Further, the apparatus CR may be configured so as to rotate the medical object MO about the longitudinal direction of extent of the medical object MO.

The system may, in addition, include a remote control unit CU that has a further representation unit 412 and a further input unit 422. The further representation unit 412 may have, for example, a monitor and/or a display and/or a projector. The further input unit 422 may include, for example, a keyboard and/or a pointing device. The further input unit 422 may be integrated into the further representation unit 412 (e.g., in the case of a capacitive and/or resistive input display). The further input unit 422 may be configured so as to acquire a user input from a remote-controlling operator. In one embodiment, the remote control unit CU may be configured, based on the detected user input, to control the apparatus, for example, by a signal CS2. The apparatus may be configured so as to adapt a positioning (e.g., position and/or orientation and/or pose) and/or a movement (e.g., translation and/or rotation) of the medical object MO in dependence upon the user input (e.g., the signal CS2). The provisioning unit PRVS may be further configured to provide PROV-SIG the signal SIG to the apparatus CR and/or the remote control unit CU. In addition, the provisioning unit PRVS may provide PROV-SIG the signal SIG to the further representation unit 412. In this case, the further representation unit 412 may be configured to display a graphic representation of the deviation and/or the warning signal (e.g., based on the signal SIG). Further, the provisioning unit may be configured so as to transmit a further signal 252 to the further representation unit 412, where the further representation unit 412 may be configured to, based on the further signal 252, display a graphic representation of the planning information PI (e.g., the planning map PABB and/or the intra-operative image data BD).

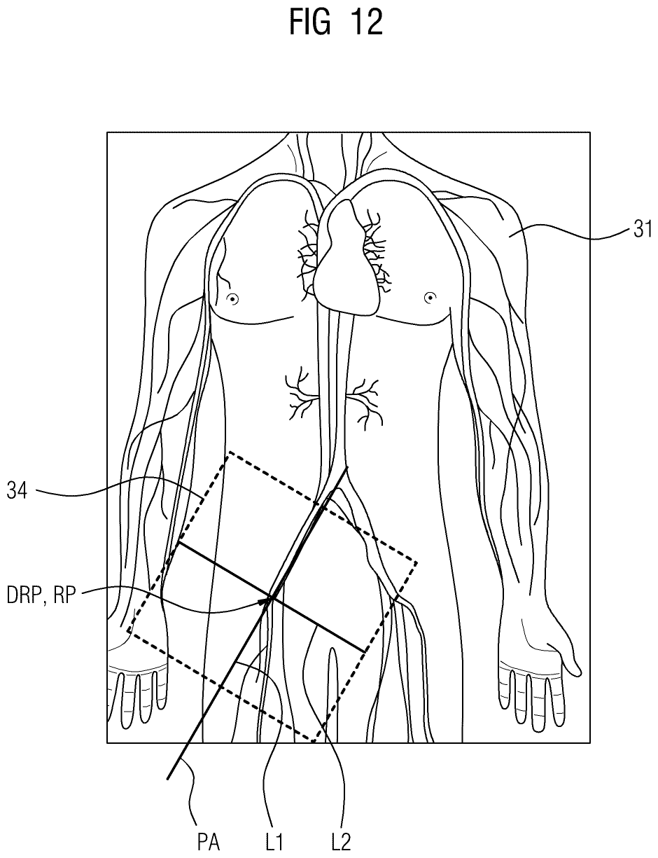

FIG. 12 shows a schematic representation of a projected light pattern, having two straight lines L1 and L2 that, for example, in the absence of the examination object 31 in the beam path between the X-ray source 33 and the detector 34, would intersect in the detector reference point DRP on the surface of the detector 34. In the operational state of the system that is illustrated in FIG. 12, the defined arrangement of X-ray source 33 (not illustrated here) and the detector 34 is repositioned RPOS-XR based on the planning information PI, such that the reference point RP of the anatomical object AO is arranged on the beam from the X-ray source 33 to the detector reference point DRP. In this case, FIG. 10 shows a view direction along the beam from the X-ray source 33 to the detector reference point DRP. The point of intersection between the two projected straight lines L1 and L2 may thus indicate the reference point RP on a surface of the examination object 31. In addition, the first straight line L1 may indicate a projection of the planning orientation PA on the surface of the examination object 31.

As assistance during puncturing and for the improved visual orientation, the detector 34 may be automatically orientated based on the planning orientation PA (e.g., the calculated puncture angle) in a plane of the patient positioning apparatus 32 (e.g., a table plane), so that the detector reference point DRP (e.g., a detector center) is arranged over the entry point IP and the detector 34 is rotated about an angle of the puncture path that lies in the table plane. The light pattern then projects both the entry point and the planning orientation onto the surface of the examination object 31. This may avoid the problem that for a “steep” angulation it is not possible for collision reasons to approach a usual “bulls-eye-view” for the puncture.

The schematic representations shown in the described figures do not depict any scale or size relationships.

Reference is made again to the fact that the methods and apparatuses described in detail above are merely exemplary embodiments that may be modified by the person skilled in the art in a wide variety of ways without leaving the scope of the invention. Further, the use of the indefinite article “a” or “an” does not exclude that the relevant features may also be present in a plurality. Likewise, the terms “unit” and “element” do not exclude that the relevant components consist of multiple interacting partial components that may also be distributed in a spatial manner where appropriate.

The elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent. Such new combinations are to be understood as forming a part of the present specification.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for monitoring an orientation of a medical object, the method comprising:
identifying planning information, the planning information having a planning orientation for the medical object with respect to a reference point on an anatomical object that is arranged within an examination object;
detecting the orientation of the medical object with respect to the reference point using an acquisition unit, wherein the acquisition unit comprises a medical imaging device, an acoustic sensor, an optical sensor, an electromagnetic sensor, or any combination thereof;
identifying a deviation between the planning orientation and the orientation of the medical object with respect to the reference point; and
providing a signal depending on the identified deviation.

2. The method of claim 1, wherein the planning information has a planning map of the examination object with the anatomical object arranged within the examination object,
wherein a planned entry point of the medical object into the anatomical object is determinable as the reference point, and
wherein identifying the planning information comprises determining the planning orientation based on a progression, an arrangement, or the progression and the arrangement of the anatomical object, which are mapped in the planning map.

3. The method of claim 2, further comprising identifying a map of at least one anatomical landmark, at least one marker structure, or a combination thereof in the planning map,
wherein the reference point, the planning orientation, or the reference point and the planning orientation are determined based on an arrangement of the at least one anatomical landmark, the at least one marker structure, or a combination thereof.

4. The method of claim 1, further comprising recording intra-operative image data using the medical imaging device,
wherein the intra-operative image data has a map of a distal portion of the medical object, which is arranged intra-operatively in the examination object, and
wherein the orientation of the medical object with respect to the reference point is detected based on the map of the distal portion of the medical object.

5. The method of claim 1, wherein the medical imaging device comprises an X-ray source and a detector that are arranged in a defined arrangement with respect to one another,
wherein the medical imaging device further comprises a light-guiding facility that is arranged on the X-ray source,
wherein the light-guiding facility is configured to project a light pattern, having at least one straight line, onto a surface of the detector so as to indicate a detector reference point, and
wherein the defined arrangement of the X-ray source and detector is repositioned based on the planning information, such that:
the reference point of the anatomical object is arranged on a beam from the X-ray source to the detector reference point; and
a projection of the planning orientation onto the surface of the detector corresponds to the at least one projected straight line.

6. The method of claim 5, wherein the light pattern has a further geometric object that is arranged on the at least one straight line, and
wherein a point of intersection of the at least one straight line with the further geometric object indicates the detector reference point.

7. The method of claim 1, further comprising detecting at least one positioning of a proximal portion of the medical object that is arranged intra-operatively outside the examination object using the acquisition unit,
wherein the orientation of the medical object with respect to the reference point is detected based on the positioning of the proximal portion.

8. The method of claim 1, further comprising:
detecting a positioning of the examination object, the anatomical object, or the examination object and the anatomical object using the acquisition unit; and
registering the planning information with the detected positioning of the examination object, the anatomical object, or the examination object and the anatomical object.

9. The method of claim 1, wherein providing the signal comprises outputting a visual warning signal, an acoustic warning signal, a haptic warning signal, or any combination thereof.

10. The method of claim 1, wherein providing the signal comprises outputting a workflow instruction so as to minimize the deviation.

11. The method of claim 1, further comprising comparing the identified deviation with a predetermined threshold value,
wherein providing the signal comprises providing the signal when, based on the comparing, the predetermined threshold value is reached, exceeded, or reached and exceeded.

12. A system for monitoring an orientation of a medical object, the system comprising:
an acquisition unit; and
a provisioning unit configured to:
identify planning information, the planning information including a planning orientation for the medical object with respect to a reference point on an anatomical object that is arranged within an examination object,
wherein the acquisition unit is configured to detect the orientation of the medical object with respect to the reference point,
wherein the acquisition unit comprises a medical imaging device, an acoustic sensor, an optical sensor, an electromagnetic sensor, or any combination thereof,
wherein the provisioning unit is configured to identify a deviation between the planning orientation and the orientation of the medical object with respect to the reference point, and
wherein the provisioning unit is configured to provide a signal depending on the identified deviation.

13. The system of claim 12, wherein the acquisition unit comprises the medical imaging device,
wherein the medical imaging device is configured to record intra-operative image data,
wherein the intra-operative image data has a map of a distal portion of the medical object that is arranged in an operational state of the system in the examination object, and
wherein the provisioning unit is further configured to detect the orientation of the medical object with respect to the reference point based on the map of the distal portion of the medical object.

14. The system of claim 12, wherein the acquisition unit comprises the medical imaging device,
wherein the medical imaging device comprises:
an X-ray source and a detector that are arranged in a defined arrangement with respect to one another;
a light-guiding facility that is arranged on the X-ray source and is configured to project a light pattern onto a surface of the detector so as to indicate a detector reference point, the light pattern having at least one straight line, and
wherein the defined arrangement of X-ray source and detector in the operational state is repositionable based on the planning information such that:
the reference point of the anatomical object is arranged on a beam from the X-ray source to the detector reference point; and
a projection of the planning orientation onto the surface of the detector corresponds to the at least one projected straight line.

15. The system of claim 12, further comprising an apparatus for robotic remote manipulation of the medical object,
wherein the provisioning unit is further configured to provide the signal to the apparatus.
